(19) United States
(12) Patent Application Publication
(10) Pub. No.: US 2024/0307587 A1
(43) Pub. Date: Sep. 19, 2024

(54) FUNCTIONAL HYDROGEL BIO-INK MODIFIED WITH BOROPHOSPHATE GLASSES

(71) Applicants: Krishna Chaitanya Reddy Kolan, Oro Valley, AZ (US); Julie A. Semon, Rolla, MO (US); Richard K. Brow, Rolla, MO (US)

(72) Inventors: Krishna Chaitanya Reddy Kolan, Oro Valley, AZ (US); Julie A. Semon, Rolla, MO (US); Richard K. Brow, Rolla, MO (US)

(73) Assignee: The Curators of the University of Missouri, Columbia, MO (US)

(21) Appl. No.: 18/608,445

(22) Filed: Mar. 18, 2024

Related U.S. Application Data
(60) Provisional application No. 63/452,904, filed on Mar. 17, 2023.

(51) Int. Cl. A61L 27/10; A61L 27/20; A61L 27/22; A61L 27/26; B29C 64/118; B29C 64/314; B29K 105/00; B29K 509/08; B29L 31/00; B33Y 10/00; B33Y 40/10; B33Y 70/00; B33Y 80/00; C09D 7/61; C09D 105/04; C09D 189/04

(52) U.S. Cl. CPC A61L 27/10; A61L 27/20; A61L 27/222; A61L 27/26; B29C 64/118; B29C 64/314; B33Y 10/00; B33Y 40/10; B33Y 70/00; B33Y 80/00; C09D 7/61; C09D 105/04; C09D 189/04; B29K 2005/00; B29K 2089/00; B29K 2105/0061; B29K 2509/08; B29L 2031/7532

(57) ABSTRACT

The present disclosure is generally directed to a bio-ink system that can be used to print cellularized scaffolds for biomedical applications. In certain configurations, the ink has rheological properties suitable for extrusion-based printing techniques and adipose stem cells infused in the ink remain viable seven days after printing. The ink may comprise a mixture of alginate hydrogels, which provide the structural integrity of the printed scaffolds, and gelatin hydrogels, which support the cell proliferation. Borophosphate glass particles are added to the hydrogel mixture where they release Ca-ions that control the viscoelastic properties of the hydrogel before and after printing. Borophosphate glasses described herein promote significantly better ASC viability than previously employed glasses.

### CROSS REFERENCE TO RELATED APPLICATIONS

This application claims the benefit of U.S. Provisional Application No. 63/452,904, filed Mar. 17, 2023, the entire contents of which are incorporated herein by reference.

### FIELD

The present disclosure is directed to hydrogel bio-inks comprising borophosphate glass particles. The present disclosure is further directed to methods of preparing the bio-ink and methods of printing a three-dimensional scaffold for biomedical applications comprising the bio-ink.

### BACKGROUND

Three-dimensional (3D) printing techniques are used to produce bio-engineered structures for biomedical applications. The materials used to print these scaffolds, the bio-inks, generally possess rheological properties that allow efficient printing of structures that maintain desired forms and functions. The bio-inks are also generally made from non-toxic materials that can be resorbed by the body and support the proliferation of stem cells present within the inks to promote tissue regeneration.

Alginate-based hydrogels have been previously employed with extrusion techniques to print 3D structures. Alginate has the requisite non-toxicity and bio-compatibility properties required for biomedical applications. Alginate-based hydrogels also generally have the rheological properties for efficient printing. To maintain the structural integrity of printed structures, alginate-based hydrogels have been previously crosslinked with divalent ions, such as calcium. Alginate does not generally support cell proliferation, so alginate-based hydrogels are typically comprised of alginate mixed with hydrogels that have RGD peptides and amide groups such as gelatin, fibrin, etc. to produce a mixed hydrogel (i.e. “alginate-based hydrogel”) that supports cell proliferation. Alginate-based hydrogel can also be infused with stem cells intended to regenerate the desired tissue and used as a bio-ink for printing 3D structures. The desired rheological properties required to print alginate-gelatin bio-inks are typically achieved by incorporating ionic solvents. However, these solvents can impact the viability of encapsulated stem cells added to the bio-inks to promote tissue regeneration.

Bioactive glasses, including the borate-based 13-93B3, have been shown to promote wound healing, although the mechanism for soft-tissue regeneration is not well-understood. Conventional borate and silicate containing bioactive glasses create local alkaline (basic) conditions when they react in aqueous solutions. This is sometimes referred to as a “pH shock” to the system/solution. The pH shock associated with the rapid release of alkali ions from the bioactive glass creates conditions that are often detrimental to cell proliferation. This is particularly observed to be detrimental under static in vitro conditions. For example, bioactive silicate glass particles (1 wt. %) have been previously added to alginate-gelatin bio-inks infused with osteogenic cells and found to reduce cell viability compared to bio-inks without the bioactive glass. Additionally, 13-93B3 glass particles have been added to alginate-gelatin bio-ink systems and infused with adipose stem cells (ASCs) before printing. This also resulted in reduced cell viability compared with bio-inks that contained no glass.

Therefore, there exists a need to produce alginate-gelatin bio-inks comprising bioactive glass that do not experience a pH shock and/or are useful for biomedical applications.

### SUMMARY

One embodiment of the present disclosure is directed to a bio-ink comprising alginate, gelatin, and borophosphate glass particles, wherein the borophosphate glass particles have a nominal molar composition of 16Na₂O-24CaO-xB₂O₃-(60-x)P₂O₅ (mol %), wherein x is an integer from 0 to 60.

A further embodiment is directed to a method of preparing a bio-ink comprising dissolving gelatin in an aqueous solution; adding borophosphate glass particles, wherein the borophosphate glass particles have a nominal molar composition of 16Na₂O-24CaO-xB₂O₃-(60-x)P₂O₅ (mol %), wherein x is an integer from 0 to 60 to the aqueous solution; stirring the aqueous solution containing borophosphate glass particles until at least a portion of the borophosphate glass particles are suspended in the aqueous solution; adding alginate to the aqueous solution having borophosphate glass particles suspended therein; and mixing the aqueous solution until at least a portion of the alginate is suspended in the aqueous solution. The disclosure is further directed to a bio-ink made according to this method.

Another aspect of the disclosure is a method of printing a three-dimensional scaffold for biomedical applications, the method comprising: loading the bio-ink prepared as described above to a syringe barrel; centrifuging the syringe until all air pockets are removed; and extruding the bio-ink from the syringe into a three-dimensional scaffold with a desired shape.

The disclosure is further directed to a three-dimensional scaffold for biomedical applications made using any of the bio-inks and/or methods described above.

Unless otherwise defined, all technical and scientific terms used herein have the same meaning as commonly understood by one of ordinary skill in the art to which this disclosure pertains. Although methods and materials similar or equivalent to those described herein can be used to practice the disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the disclosure are set forth in the accompanying drawings and the description below. Other features, objects, and advantages of the disclosure will be apparent from the description and drawings, and from the claims.

### BRIEF DESCRIPTION OF THE DRAWINGS

FIGS. 1A-1B depict the pH change over time for borophosphate glass particles, having varying boron content, dissolved in water (FIG. 1A) and in cell culture medium (FIG. 1B).

FIGS. 2A-2B depict the pH change of hydrogels with differing concentrations and identities of borophosphate glass particles at 1 and 7 days. FIG. 2A reports results at 2 wt. %, and FIG. 2B reports results at 10 wt. %.

FIGS. 3A-3D depict the ion releases rates for certain elements of glass particles in hydrogels at 1 and 7 days at concentrations of 1.25, 2.5, 5, and 10 wt. %. FIG. 3A illustrates the release rate for 13-93B3. FIG. 3B illustrates the release rate for borophosphate glass particles having a nominal molar composition of 16Na₂O-24CaO-xB₂O₃-(60-x)P₂O₅ (mol %), wherein x is 20. FIG. 3C illustrates the release rate for borophosphate glass particles having a nominal molar composition of 16Na₂O-24CaO-xB₂O₃-(60-x)P₂O₅ (mol %), wherein x is 40. FIG. 3D illustrates the release rate for borophosphate glass particles having a nominal molar composition of 16Na₂O-24CaO-xB₂O₃-(60-x)P₂O₅ (mol %), wherein x is 60.

FIGS. 4A-4D depict the concentrations of Ca and Pions released to the gels from the borophosphate glass particles after 1 and 7 days. FIG. 4A depicts Ca-ion concentrations in the hydrogels with 2 wt. % glass particles. FIG. 4B depicts Ca-ion concentrations in the hydrogels with 10 wt. % glass particles. FIG. 4C depicts P-ion concentrations in the hydrogels with 2 wt. % glass particles. FIG. 4D depicts P-ion concentrations in the hydrogels with 10 wt. % glass particles.

FIGS. 5A-5C depict the shear rate and viscosity relationship of alginate hydrogels, gelatin hydrogels, alginate-gelatin hydrogels, and hydrogels comprising borophosphate glass particles. FIG. 5A compares gelatin hydrogels, alginate hydrogels, and alginate-gelatin hydrogels. FIG. 5B compares hydrogel samples made with 1.25 wt. % X0, X20, X40, and X60 glass particles. FIG. 5C compares hydrogel samples made with 10 wt. % X0, X20, X40, and X60 glass particles.

FIG. 6 depicts hydrogel recovery with borophosphate glass particles, shown as viscosity versus time. Recovery time is directly related to glass dissolution rate and likely Ca²⁺ion release, and recovery is related to how filament behaves after printing.

FIGS. 7A-7C depict gelatin release from a printed scaffold. FIG. 7A depicts cumulative gelatin release over time. FIG. 7B depicts dissolution of gelatin-fractions in water from undoped hydrogels (Alg+Gel) and hydrogels doped with 1.25 wt. %, 2.5 wt. %, 5 wt. %, and 10 wt. % of X40 glass at day 7. FIG. 7C compares a reference, day 1, and day 7 data.

FIGS. 8A-8C depict elastic and viscous modulus of the different components of the hydrogel. FIG. 8A depicts the results for gelatin hydrogels. FIG. 8B depicts the results for alginate hydrogels. FIG. 8C depicts the results for alginate-gelatin hydrogels.

FIGS. 9A-9D depict elastic and viscous modulus of hydrogels with different glass particles. FIG. 9A depicts the results for hydrogels with X0. FIG. 9B depicts the results for hydrogels with X20. FIG. 9C depicts the results for hydrogels with X40. FIG. 9D depicts the results for hydrogels with X60.

FIGS. 10A-10D depict internal cross section of hydrogels with and without crosslinker or X40 glass particles. FIG. 10A depicts a crosslinked alginate-gelatin hydrogel with X40 glass particles. FIG. 10B depicts an uncrosslinked alginate-gelatin hydrogel with X40 glass particles. FIG. 10C depicts a crosslinked alginate-gelatin hydrogel without glass particles. FIG. 10D depicts an uncrosslinked alginate-gelatin hydrogel without glass particles. Close-view photographs were taken either post mixing/crosslinking and compared to post freeze-dried samples.

FIGS. 11A-11D depict images of hydrogels with or without X40 and with or without crosslinking. FIG. 11A depicts a crosslinked alginate-gelatin hydrogel with X40 glass particles. FIG. 11B depicts an uncrosslinked alginate-gelatin hydrogel with X40 glass particles. FIG. 11C depicts a crosslinked alginate-gelatin hydrogel without glass particles. FIG. 11D depicts an uncrosslinked alginate-gelatin hydrogel without glass particles.

FIGS. 12A-12C depict scaffold fabrication using bio-inks comprising borophosphate glass particles. Grid scaffolds were printed from bio-ink hydrogels doped with X20 glass particles (FIG. 12A), X40 glass particles (FIG. 12B), and X60 glass particles (FIG. 12C).

FIG. 13 depicts cell viability after 7 days in static culture for scaffolds printed with bio-ink hydrogels doped with different borophosphate glass particles. Images in the top and middle rows are separated into green (live cells) and red channel (dead cells) images showing only that color of cells. Color images in the bottom row show both live and dead cells.

FIG. 14 depicts cell viability measurements for scaffolds printed with undoped bio-ink hydrogels and bio-ink hydrogels doped with borophosphate glass particles. Measurements were taken immediately after printing, then one and seven days after printing. For each day, the bars show (left to right): no glass, X20, X0, X40, and B3 =X60.

### DETAILED DESCRIPTION

The instant disclosure is directed in certain embodiments to a bioactive glass that releases ions similar to those released by the borate-based glass 13-93B3, but in a way that does not create the basic “pH shock” conditions in aqueous environments. By varying the phosphate-to-borate ratio in bioactive glasses that are Na-Ca-borophosphate glasses, local pH conditions can be controllably adjusted from acidic to basic.

For example, the inventors have discovered that a borophosphate glass having the nominal molar composition 16Na₂O-24CaO-xB₂O₃-(60-x)P₂O₅ is observed to exhibit acidic (pH<4) conditions when glasses with x=0, 10, and 20 are dissolved in water, basic conditions when glasses with x=60 are dissolved, and neutral conditions (6<pH<7) when glasses with x=30 or x=40 are dissolved. Similar pH shifts, although less in magnitude, occur when the same glasses are reacted in simulated body fluid.

The inventors have surprisingly discovered that the incorporation of pH neutral glass particles into an alginate-gelatin system creates a particularly suitable bio-ink. This bio-ink comprises borophosphate glass particles that release desired ionic species that stimulate desired cellular responses and that control the bio-ink viscosity to optimize printing. The advantage of these formulations is that the borophosphate glass particles do not induce the “pH shock” effect that has been shown to be detrimental to cell viability in other bio-ink formulations. This functional PH neutral bio-ink can be used to fabricate human tissue scaffolds and has the potential in the clinic to repair and replace damaged or diseased tissue. It also has the potential to be used by many researchers in the ever growing field of human organoid development, which creates improved models of human tissues and organs for drug development, disease modeling, and basic cellular and molecular studies. By using this bio-ink to fabricate human tissues, this bio-ink would be of interest to clinicians and scientists who study cancer, immunology, aging, diabetes, regenerative medicine, as well as many other fields. In certain aspects of the present disclosure, the system is tunable as to pH. That is, different glass types can be used to produce pH values suitable for different contexts.

Certain embodiments of the present disclosure are directed to a bio-ink comprising alginate, gelatin, and borophosphate glass particles, wherein the borophosphate glass particles have a nominal molar composition of 16Na₂O-24CaO-xB₂O₃-(60-x)P₂O₅ (mol %), wherein x is an integer from 0 to 60. In some embodiments, the bio-ink can have rheological properties suitable for extrusion-based printing techniques.

Further embodiments of the present disclosure are directed to a method of preparing a bio-ink, the method comprising: a) dissolving gelatin in an aqueous solution (e.g., at 40° C.) with stirring; b) adding the borophosphate glass particles to the aqueous solution; c) stirring the aqueous solution until at least a portion of the borophosphate glass particles are suspended in the aqueous solution; d) adding alginate to the aqueous solution having borophosphate glass particles suspended therein; and e) mixing the aqueous solution until at least a portion of the alginate is suspended in the aqueous solution. In some embodiments, the bio-ink can be used for printing a three-dimensional scaffold for biomedical applications. In one embodiment, the printing comprises a method comprising: a) loading the bio-ink prepared as described above to a syringe barrel; b) centrifuging the syringe until all air pockets are removed; and c) extruding the bio-ink from the syringe into a three-dimensional scaffold with a desired shape. In some embodiments, the three-dimensional scaffold can be used for biomedical applications.

One aspect of the present disclosure is directed to a bio-ink comprising alginate, gelatin, and borophosphate glass particles, wherein the borophosphate glass particles have a nominal molar composition of 16Na₂O-24CaO-xB₂O₃-(60-x)P₂O₅ (mol %), and wherein x is an integer from 0 to 60. In certain embodiments, the bio-ink can have rheological properties suitable for extrusion-based printing techniques.

In certain embodiments of the present disclosure, the bio-ink comprises of alginate, gelatin, borophosphate glass particles, and a solvent, wherein the borophosphate glass particles have a nominal molar composition of 16Na₂O-24CaO-xB₂O₃-(60-x)P₂O₅ (mol %), wherein x is an integer from 0 to 60.

In some embodiments, the alginate may be sodium alginate.

In various embodiments, the alginate-gelatin hydrogels, bio-ink hydrogels, etc. described herein may be an alginate-natural hydrogel or alginate-synthetic hydrogel. For example, the hydrogel may be an alginate-natural hydrogel, wherein the natural component is selected from the group consisting of gelatin, agarose, collagen, hyaluronic acid, and combinations thereof. In other embodiments, the hydrogel may be an alginate-synthetic hydrogel, wherein the synthetic component is selected from the group consisting of polyethylene glycol (PEG), pluronic F-127, and combinations thereof.

In some embodiments, the alginate may be from about 0.1% to about 15 w/v %, from about 0.5% to about 12 w/v %, from about 1% to about 10 w/v %, from about 2 to about 7 w/v %, from about 2 to about 5 w/v %, or from about 3 to about 4 w/v % of the bio-ink, wherein w/v % is weight per volume of total bio-ink.

In certain embodiments, for a borophosphate glass particle having a nominal molar composition of 16Na₂O-24CaO-xB₂O₃-(60-x)P₂O₅ (mol %), x may be from about 0 to about 60, from about 5 to about 60, from about 10 to about 60, from about 15 to about 60, from about 20 to about 60, from about 25 to about 60, from about 30 to about 60, from about 30 to about 55, from about 30 to about 50, from about 30 to about 45, or from about 30 to about 40. In one embodiment, x is from about 0 to about 20, from about 0 to about 15, from about 0 to about 10, or from about 0 to about 5. In another embodiment, x is from about 40 to about 60, from about 45 to about 60, from about 50 to about 60, or from about 55 to about 60.

In certain embodiments, the borophosphate glass particles are from about 10 μm to about 500 μm, from about 30 μm to about 400 μm, from about 50 μm to about 300 μm, from about 50 μm to about 200 μm, from about 75 μm to about 150 μm, or from about 100 μm to about 125 μm in diameter.

In certain embodiments, the borophosphate glass particles represent from 0.1% to about 20 w/v %, from about 1% to about 20 w/v %, from about 2% to about 18 w/v %, from about 4% to about 16 w/v %, from about 6% to about 14 w/v %, from about 8 to about 12 w/v %, or from about 9% to about 11 w/v % of the bio-ink, wherein w/v % is weight per volume of total bio-ink.

Although discussion herein may be directed to borophosphate glass particles, it will be understood that the present disclosure also encompasses the use of borophosphate glass and hydrogels or bio-inks comprising borophosphate glass in any other suitable form. For example, borophosphate glass powder, borophosphate glass that has been dissolved or suspended in composition, etc.

In some embodiments, the gelatin may be from about 0.1% to about 15 w/v %, from about 0.5% to about 12 w/v %, from about 1% to about 10 w/v %, from about 2 to about 7 w/v %, from about 2 to about 5 w/v %, or from about 3 to about 4 w/v % of the bio-ink, wherein w/v % is weight per volume of total bio-ink.

In certain embodiments, the gelatin is from about 0.1% to about 15 w/v %, from about 0.5% to about 12 w/v %, from about 1% to about 10 w/v %, from about 2 to about 7 w/v %, from about 2 to about 5 w/v %, or from about 3 to about 4 w/v %; the alginate is from about 0.1% to about 15 w/v %, from about 0.5% to about 12 w/v %, from about 1% to about 10 w/v %, from about 2 to about 7 w/v %, from about 2 to about 5 w/v %, or from about 3 to about 4 w/v %; and the borophosphate glass particles are from about 0.1% to about 20 w/v %, from about 1% to about 20 w/v %, from about 2% to about 18 w/v %, from about 4% to about 16 w/v %, from about 6% to about 14 w/v %, from about 8 to about 12 w/v %, or from about 9% to about 11w/v % of the bio-ink, wherein w/v % is weight per volume of total bio-ink.

In one embodiment, the gelatin is about 3 w/v %, the alginate is about 3 w/v %, the borophosphate glass particles are about 10 w/v % of the bio-ink, and x is about 30 to about 40. In certain embodiments, a bio-ink of this composition has a pH of from about 6 to about 7.

In some embodiments, the solvent may be selected from the group consisting of water, Dulbecco's Modified Eagle Medium (DMEM), another cell culture medium, and combinations thereof. For example, the solvent may be selected from the group consisting of DMEM, minimal essential medium Eagle-alpha modification (aMEM), DMEM: F12 Nutrient Mixture (DMEM:F12), and combinations thereof. In certain embodiments, the solvent may be any solvent that is suitable for dissolving alginate, gelatin, and borophosphate glass particles.

In certain embodiments, the bio-ink may further comprise mammalian cells. For example, the mammalian cells can be selected from the group consisting of adult stem cells (e.g., adipose stem cells), fibroblast cells, cancer cells, and any other suitable cell type known in the art.

In some embodiments, the bio-ink may further comprise a cell culture medium. For example, the cell culture medium can be selected from the group consisting of Dulbecco's Modified Eagle Medium (DMEM), minimal essential medium Eagle-alpha modification (αMEM), DMEM: F12 Nutrient Mixture (DMEM:F12), and any other suitable cell culture medium known in the art.

In certain embodiments, the bio-ink of the present disclosure may further comprise a crosslinker. For example, the crosslinker can be selected from the group consisting of CaCl₂, thrombin, fetal bovine serum (FBS), and combinations thereof. In other embodiments, the bio-ink may comprise no additional crosslinker.

In certain embodiments, the pH of the bio-ink may be acidic, neutral, or basic. In one embodiment, the pH may be acidic or neutral. In some embodiments, the pH may be about from 2 to about 6, from about 3 to about 6, from about 4 to about 6, from about 5 to about 6, or from about 5.5 to about 6. In other embodiments, the pH may be from about 6 to about 7, from about 6 to about 6.8, from about 6 to about 6.4, from about 6 to about 6.2, from about 6.2 to about 7, from about 6.4 to about 7, from about 6.6 to about 7, from about 6.8 to about 7, or about 7. In other embodiments, the pH may be from about 7 to about 12, from about 7 to about 11, from about 7 to about 10, from about 7 to about 9, from about 7 to about 8, or from about 7.5 to about 8.

In various embodiments, the borophosphate glass particles have a nominal molar composition of 16Na₂O-24CaO-xB₂O₃-(60-x)P₂O₅ (mol %), x is from about 0 to about 20, and the pH is less than about 4. In other embodiments, x is from about 30 to about 40, and the pH is about 6 to about 7. In still further embodiments, x is from about 40 to about 60 and the pH is greater than 7.

Certain aspects of the present disclosure are further directed to methods of preparing a bio-ink. For example, preparing a bio-ink comprising alginate, gelatin, and borophosphate glass particles, wherein the borophosphate glass particles have a nominal molar composition of 16Na₂O-24CaO-xB₂O₃-(60-x)P₂O₅ (mol %), wherein x is an integer from 0 to 60.

In one embodiment, the method comprises: a) dissolving gelatin in an aqueous solution (e.g., by stirring); b) adding the borophosphate glass particles to the aqueous solution; c) stirring the aqueous solution containing borophosphate glass particles until the borophosphate glass particles are uniformly suspended (or dissolved) in the aqueous solution; d) adding alginate to the suspended solution; and e) mixing the combination until the alginate is uniformly suspended in the aqueous solution. In certain embodiments, stirring and/or mixing can comprise placing the components in a glass beaker and using a magnetic stir bar and hot plate. Although various aspects are noted as “suspended,” it will be understood that this intends to reference, in one embodiment, solutions wherein at least a portion of the component is suspended.

In some embodiments, the method may comprise: a) dissolving gelatin in an aqueous solution; b) adding borophosphate glass particles, wherein the borophosphate glass particles have a nominal molar composition of 16Na₂O-24CaO-xB₂O₃-(60-x)P2O₅ (mol %), wherein x is an integer from 0 to 60 to the aqueous solution; c) stirring the aqueous solution containing borophosphate glass particles until the borophosphate glass particles are suspended in the aqueous solution; d) adding alginate to the aqueous solution having borophosphate glass particles suspended therein; and e) mixing the aqueous solution until the alginate is suspended in the aqueous solution. In some embodiments, the bio-ink can be used for printing a three-dimensional scaffold for biomedical applications. In one embodiment, the printing comprises a method comprising: a) loading the bio-ink prepared as described above to a syringe barrel; b) centrifuging the syringe until all air pockets are removed; and c) extruding the bio-ink from the syringe into a three-dimensional scaffold with a desired shape. In some embodiments, the three-dimensional scaffold can be used for biomedical applications.

In certain embodiments, the method further comprises step f) wherein mammalian cells are added to the solution and stirred until the cells are evenly distributed. For example, the mammalian cells can comprise adult stem cells (e.g., adipose stem cells), fibroblast cells, cancer cells, or any other suitable cell type known in the art. In some embodiments, the mammalian cells have a concentration of from about 1.0×10³ cells/m to about 1.0×10¹⁰ cells/mL or from about 1.0×10⁵ cells/m to about 1.0×10⁷ cells/mL.

In certain embodiments, the method may comprise preparing a bio-ink comprising a cell culture medium. For example, a cell culture medium may be selected from the group consisting of Dulbecco's Modified Eagle Medium (DMEM), minimal essential medium Eagle—alpha modification (αMEM), DMEM: F12 Nutrient Mixture (DMEM:F12), and combinations thereof.

In various embodiments, the method may be conducted at a temperature of from about 20° C. to about 60° C. or from about 30° C. to about 50° C. In one embodiment, the temperature may be about 40° C.

The identity of the borophosphate glass particles, gelatin, alginate, solvents, etc. may be as described in further detail above.

Certain aspects of the present disclosure are further directed to a bio-ink made according to this method.

Another aspect of the present disclosure is directed to a method of printing the bio-ink for biomedical applications. Although reference is made herein to preparing a three-dimensional scaffold from the bio-ink, it will be understood that the bio-ink may be used as described herein to form or print any desirable shape or product.

In certain embodiments, the method comprising: a) loading the bio-ink prepared as described above to a syringe barrel; b) centrifuging the syringe until all air pockets are removed; and c) extruding the bio-ink from the syringe. For example, the bio-ink may be extruded, in one embodiment, into a three-dimensional scaffold with a desired shape.

In some embodiments, step c) can be performed using a 3D printer. In other embodiments, the extruding step may be conducted by any suitable method for extrusion.

In certain embodiments, the shape of the three-dimensional scaffold can be any regular shape. For example, a rectangle, square, circle, or oval. The shape can alternatively be irregular and/or designed to fit into a specific cavity. In some embodiments, the shape may comprise one or more holes. In one embodiment, the major axis of the shape can be from about 0.1 mm to about 100 mm, from about 0.5 mm to about 50 mm, from about 1 mm to about 25 mm, or from about 5 mm to about 20 mm. In one embodiment, the shape is a rectangle about 15 mm long, about 15 mm wide, and about 1 mm high.

The three-dimensional scaffold of the present disclosure may comprise multiple layers, wherein adjacent layers are printed in orthogonal orientation to each other. For example, the three-dimensional scaffold may comprise from about one to about fifty layers, from about one to about twenty layers, or from about five to about ten layers. In other embodiment, the three-dimensional scaffold may comprise about one layer, about two layers, about three layers, about four layers, about five layers, about six layers, about seven layers, about eight layers, about nine layers, or about ten layers.

The disclosure is further directed, in certain embodiments, to a three-dimensional scaffold for biomedical applications made using any of the bio-inks and/or methods described above.

Various embodiments of the hydrogels, bio-inks, or printed bio-inks (e.g., three-dimensional scaffolds) described herein comprise mammalian cells or other components that are pH sensitive. One aspect of the present invention is directed to preparing a hydrogel, bio-ink, or printed bio-ink that maintains cell viability and/or maintains a suitable pH for a period of time. For example, the hydrogels, bio-inks, or printed bio-inks described herein may comprise mammalian cells or other components that remain viable for at least about 1 day, at least about 2 days, at least about 3 days, at least about 4 days, at least about 5 days, at least about 6 days, at least about 1 week, or at least about 2 weeks after preparation. In one embodiment, a printed bio-ink comprises adipose cells that remain viable for at least about 1 day, at least about 2 days, at least about 3 days, at least about 4 days, at least about 5 days, at least about 6 days, at least about 1 week, or at least about 2 weeks after printing.

It is to be understood that while the disclosure has been described in conjunction with the detailed description thereof, the preceding description is intended to illustrate and not limit the scope of the disclosure, which is defined by the scope of the appended claims. Other aspects, advantages, and modifications are within the scope of the following claims.

### EXAMPLES

The disclosure will be further described in the following examples, which are to be considered as merely illustrative, and are not intended to limit the scope of this disclosure.

### Example 1: Preparing Materials for Hydrogel Composition

### Glass Preparation

Certain bioactive glasses were prepared for use in the examples set forth below.

Four bioactive borophosphate glasses were prepared having the nominal molar composition 16Na₂O-24CaO-xB₂O₃-(60-x)P₂O₅ (mol %), where x was 0, 20, 40, and 60. A borate bioactive glass 1393-B3 (B3), with the nominal composition 6Na₂O, 12K₂O, 5MgO, 20CaO, 4P₂O₅, 53B₂O₂ (wt. %), was also prepared.

Reagent grade batch materials for each composition were calcined at 300° C. for at least 4 hours and then melted for an hour in platinum crucibles at 1000-1150° C., depending on composition. The melted materials were stirred after 30 minutes with a platinum rod, and then quenched in graphite molds. The quenched samples were annealed at 350° C. for one hour then allowed to cool to room temperature and stored in a vacuum desiccator until use. The resulting glasses were analyzed by x-ray diffraction, using a PANalytical X′Pert Multipurpose diffractometer with a Cu K-a source and a PIXcel detector. All glasses were found to be amorphous.

The results are reported below in Table 1. Nominal compositions are reported in mol %.

TABLE 1
Bioactive glasses.

| Bioactive | | | | | | | |
|---|---|---|---|---|---|---|---|
| Glass | SiO₂ | Na₂O | K₂O | MgO | CaO | P₂O₅ | B₂O₃ |
| 13-93B3 | 0 | 6.0 | 12.0 | 5.0 | 20.0 | 4.0 | 53.0 |
| X0 | 0 | 16 | 0 | 0 | 24 | 60 | 0 |
| X20 | 0 | 16 | 0 | 0 | 24 | 40 | 20 |
| X40 | 0 | 16 | 0 | 0 | 24 | 20 | 40 |
| X60 | 0 | 16 | 0 | 0 | 24 | 0 | 60 |

### Hydrogel Preparation

A gelatin-alginate hydrogel was prepared. Gelatin (Type B, commercially available from Sigma-Aldrich, St. Louis, MO, USA), sodium alginate (commercially available from Sigma-Aldrich, St. Louis, MO, USA), and bioactive glasses noted above were first UV sterilized for 5 min.

Gelatin in 3 wt/vol % (0.3 g in 10 mL) was dissolved in Dulbecco's Modified Eagle Medium (DMEM) (Gibco, commercially available from ThermoFisher Scientific, MA, USA) in a glass beaker at ˜40° C. while being magnetically stirred at 200 RPM. Once the gelatin was fully dissolved, the bioactive glass particles noted above, in an amount equal to 10 wt. % of total hydrogel material, were added to the gelatin solution and stirred to obtain a uniform suspension of glass particles. Sodium alginate was then added to the suspension and mixed overnight to produce a gelatin-alginate hydrogel comprising bioactive glass (i.e. bio-ink).

The resulting gelatin-alginate hydrogel comprising bioactive glass was transferred to a 3 mL Loctite® Henkel syringe barrel, centrifuged to remove air pockets, and attached with 25 G (100 μm) tips (SmoothFlow Tapered, commercially available from Nordson EFD, Westlake, OH, USA) for fabrication and use, as discussed in further detail below.

### Example 2: Ph Change, Glass Dissolution, and Ion Release

### Ph Change From Glass Particle Dissolution

A test was conducted to determine the pH change, glass dissolution and ion concentrations in dissolution solutions containing glass particles.

Nylon mesh bags having opening of 45 μm were cach filed with 150±1 mg of glass particles (75-150 microns) and sealed. The mesh bags were then immersed in 50 ml of either (1) deionized water or (2) cell culture media (stored in high density polyethylene centrifuge tubes in a shaker bath at 37° C.). The immersed bags were removed after 24 hours, dried and weighed. These pH and ion concentration measurements were done in triplicate, and the average values were reported. Glass dissolution and ion concentrations in the dissolution solutions were analyzed using Inductively Coupled Plasma-Optical Emission Spectroscopy (ICP-OES), using a Perkin-Elmer Avio 200.

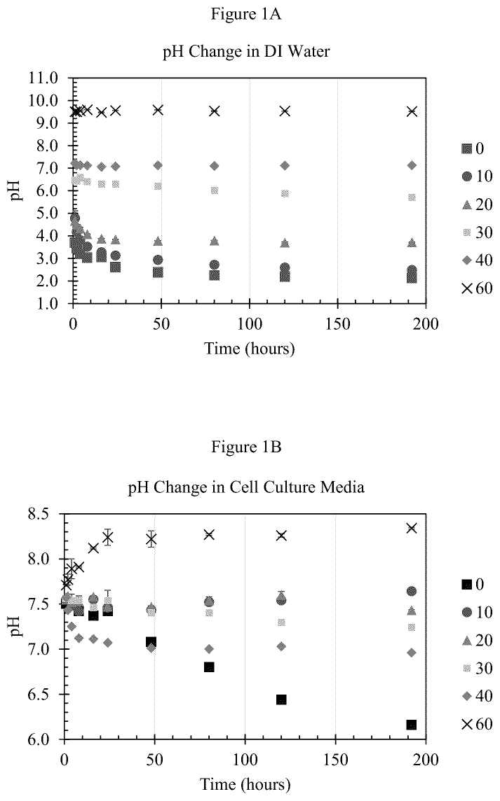

The results showing the effect of borophosphate glass particles on solution pH in DI water and cell culture medium are shown in FIGS. 1A and 1B, respectively. The varied boron content in the borophosphate glass series controls the dissolution rate of glass particles and thereby pH. There is a clear trend as phosphate rich glasses, X20 and X0, produce more acidic solutions, and borate rich glasses, X40 and X60, produce more basic solutions, although X40 is more pH-neutral than basic. This corresponds to the dissolution rate at which Ca²⁺ is released from the glass particles (X20<X0<X40<X60).

### Ph Change From Glass Particles In Media

Next, the pH change in surrounding media of varying wt. % of borophosphate glass hydrogels at 1 and 7 days was measured.

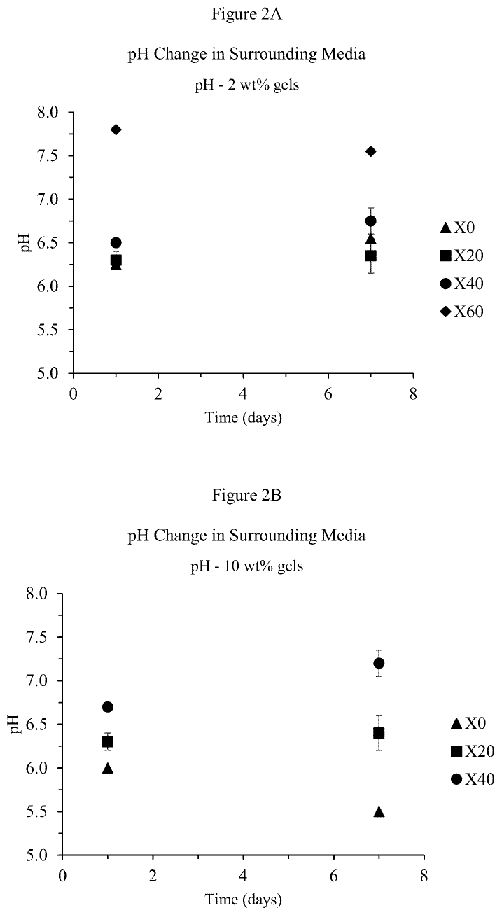

As shown in FIGS. 2A-2B, hydrogels with lower concentrations of glass particles and with a higher amount of phosphate in the glass particles stayed around the same acidic pH values in a buffered simulated body fluid (SBF) solution (˜6) through day 7.

At 10 wt. %, XO glass particles had the lowest pH values and continued to decrease at Day 7. In contrast, the X20 glass particle compositions had increased pH over time. X40 glass particle compositions continued to stay pH-neutral, even though they had a higher borate content, which was consistent with prior research. The pH increase was significant for the basic borate glass (X60 glass) particles at 2 wt. %. Any pH approaching 8 is generally considered to be too basic for cells and will have a negative effect on cell viability. Furthermore, hydrogels with 10 wt. % X60 glass particles proved too difficult to print (as explained in further detail below), as X60 glass particles dissolved too fast and did not produce the uniform hydrogel needed for printing.

### Ion Release Rates

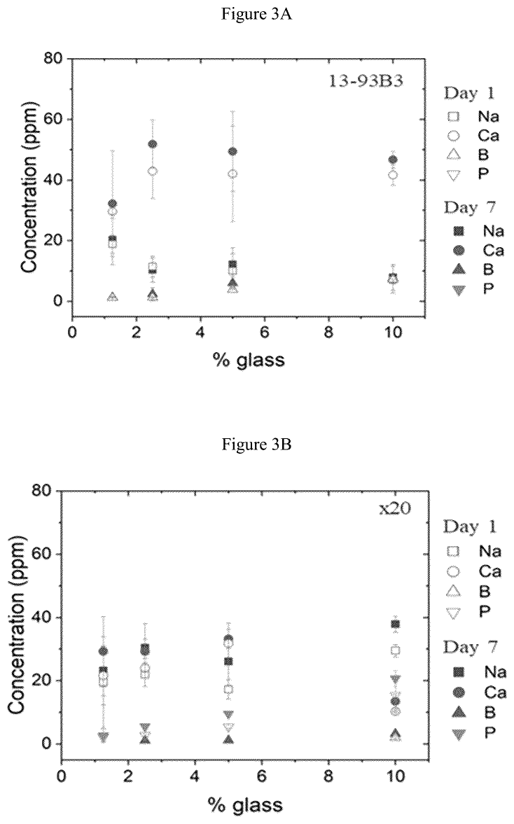

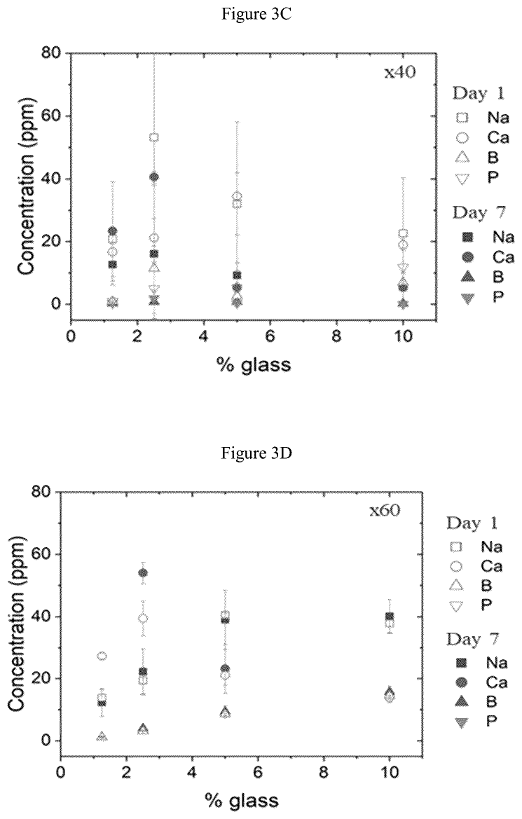

Individual ion release rates were also measured. The ion release rates of Na, Ca, B, and P at day 1 and 7 days were measured for the hydrogels described above doped with X20, X40, and X60. These hydrogels were compared to 13-93B3. All samples were tested at concentrations of 1.25, 2.5, 5, and 10 wt. %. The results, reported in FIGS. 3A-3D, illustrate that most of the glass groups and concentrations follow the trend of increased release of each ion over time. FIG. 3A illustrates the release rate for 13-93B3. FIG. 3B illustrates the release rate for borophosphate glass particles having a nominal molar composition of 16Na₂O-24CaO-xB₂O₃-(60-x)P₂O₅ (mol %), wherein x is 20. FIG. 3C illustrates the release rate for borophosphate glass particles having a nominal molar composition of 16Na₂O-24CaO-xB₂O₃-(60-x)P₂O₅ (mol %), wherein x is 40. FIG. 3D illustrates the release rate for borophosphate glass particles having a nominal molar composition of 16Na₂O-24CaO-xB₂O₃-(60-x)P₂O₅ (mol %), wherein x is 60.

X40 at 1.25 and 2.5 wt. % exhibited the trend of increased release of each ion over time, but at 5 and 10 wt. % X40 suddenly had a decrease in release for each ion. X40 also had a larger difference between day 1 and 7 samples for Na and Ca. At 2.5 wt. % of the difference was about 40 ppm for Na.

13-93B3 released the most Ca overall for any glass particle concentration but does not have a large change between time points.

X20 did not have a significant difference in ion release between 1 and 7 days for each concentration due to its slow-release dissolution rate.

X60 behaved like 13-93B3 at lower concentrations, but at 5 and 10 wt. % released less Ca and more Na.

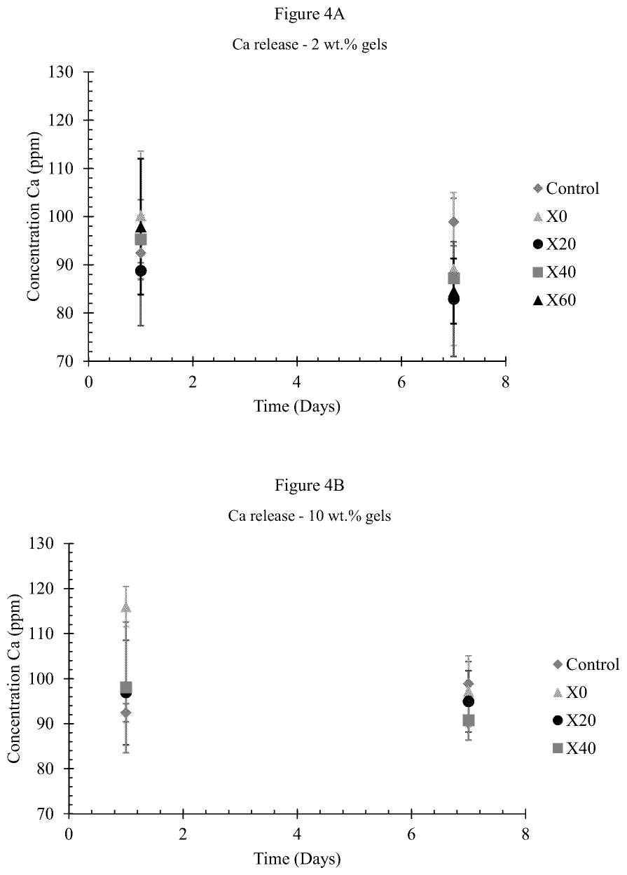

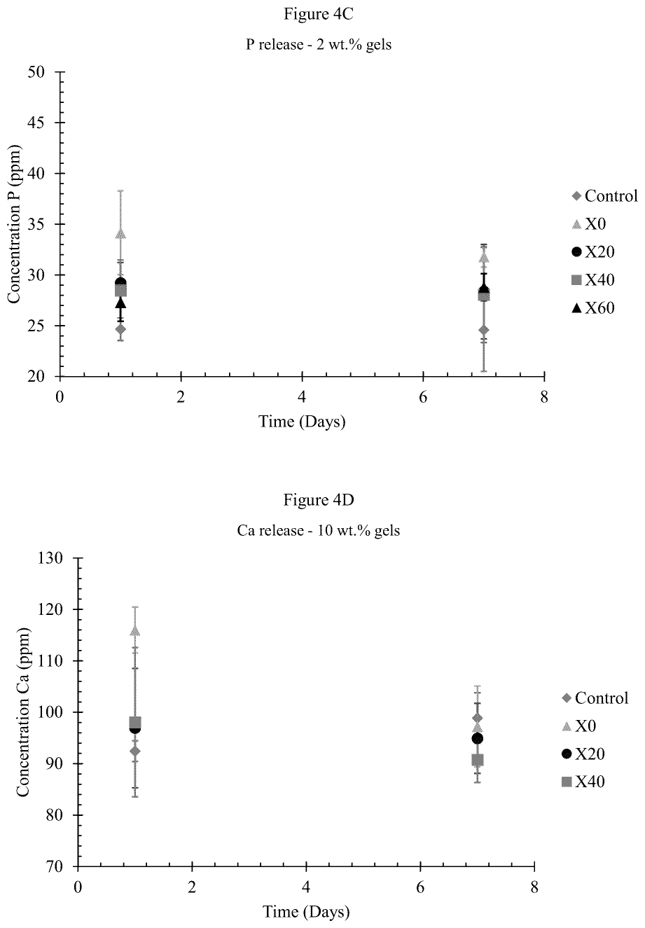

FIG. 4A-4D illustrate the Ca and P release rates for the X series glass particles and a hydrogel by itself (i.e. no glass). FIG. 4A depicts Ca release in 2 wt. % hydrogels. FIG. 4B depicts Ca release in 10 wt. % hydrogels. FIG. 4C depicts P release in 2 wt. % hydrogels. FIG. 4D depicts P release in 10 wt. % hydrogels.

In the glass-free hydrogel, there was an upward trend in Ca release from 1 to 7 days at both concentrations. This could be due to alginate dissolving and/or gelatin being released from the samples.

For the rest of the glasses, X0 has the highest release of Ca and P in every scenario. This was unexpected. A further unexpected result was the upward trend for all of the X series glass particles over time in 10 wt. % hydrogels, except X60 (which did not contain any P). The dissolution trend of X20<X0<X40<X60 was expected, as the fast-reacting glass particles have a higher decrease and release more ions between Day 1 and Day 7 as compared to X20. Other research has shown that in SBF, X20 dissolves about 30 times slower than X0, which dissolves about 10 times slower than X40, and X40 dissolves about half as fast as X60.

### Example 3: Viscoelastic Behavior of Borophosphate Glasses

An ideal bio-ink should demonstrate two properties to guarantee high shape fidelity and printing accuracy: 1) the ability to exhibit a shear-thinning behavior upon the application of a deforming force; and 2) the ability to exhibit a rapid increase in viscosity after the removal of a force.

A mechanical test was conducted to determine the viscosity of compositions containing varying concentrations of borophosphate glass particles.

Hydrogels were prepared in deionized water comprising gelatin (3 w/v %) and alginate (3 w/v %), resulting in an alginate-gelatin hydrogel having a concentration of 6 w/v %. Varying concentrations of borophosphate glass particles were added to the alginate-gelatin hydrogel.

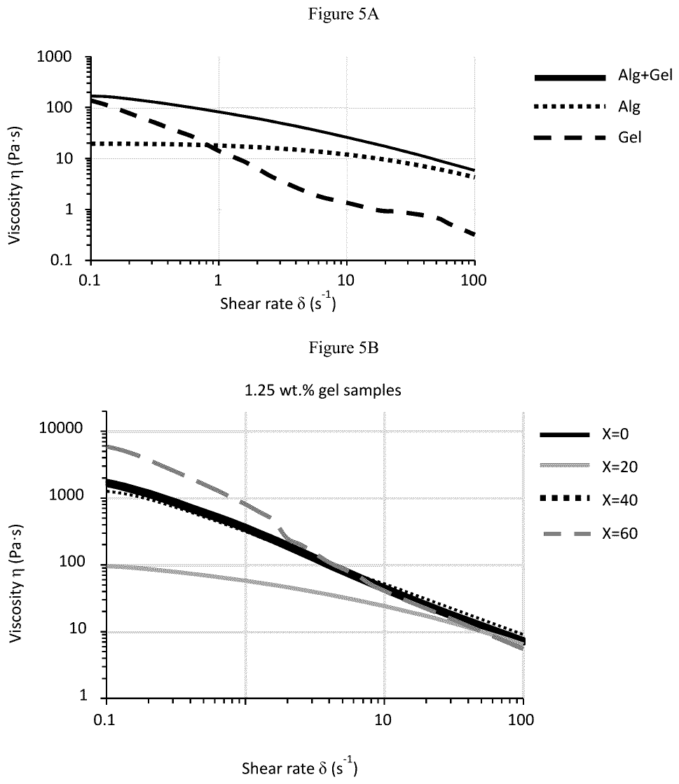

The alginate-gelatin hydrogels with borophosphate glass particles, and an alginate-gelatin hydrogen without glass, were tested for viscosity using a Kinexus rheometer (commercially available from Malvern Panalytical, Westborough, MA, USA) with a parallel plate set-up. A gap of 0.5 mm was set between plates, and measurements were conducted at room temperature. Hydrogels were subjected to increasing shear rates from 0.01 to 1000 s⁻¹, with the results reported in FIGS. 5B and 5C. Data points below 0.01 s1 shear rate were not reported due to the instability of the hydrogel at low shear rates. Measurements were repeated to confirm the validity of the data. A fresh scoop of hydrogel was loaded each time to test viscosity, recovery, yield strength, etc.

In water and in SBF the dissolution rates of these glass particles increase in the order X20<X0<X40<X60. In FIG. 5C, when 10 wt. % glass particles were added to an alginate-gelatin mixture, the viscosity of this composite hydrogel increased in the same order, from about 25 Pa-s (shear rate of 1 s⁻1) for the X20 glass particles to 2000 Pa-s for the X60 glass particles. Therefore, increasing the rate at which Ca-ions are released to the hydrogel is thought to increase the extent of crosslinking of the alginate, and thereby the viscosity of the hydrogel. Alginate-gelatin hydrogels with borophosphate glass particles also had increased viscosity compared to gelatin or alginate hydrogels alone, as shown in FIG. 5A.

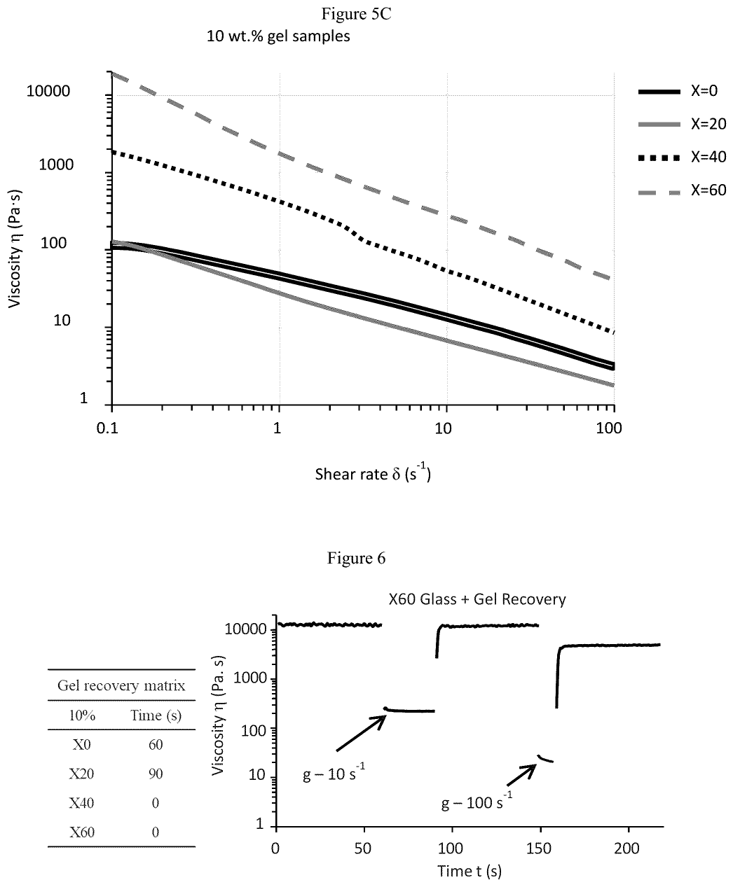

FIG. 6 illustrates that the hydrogels doped with borophosphate glass particles possess the shear-thinning viscoelastic properties desired for printing: low viscosity under the high shear-rates associated with printing, then high viscosity when the shearing conditions were removed. That is, that structures that are printed under the high-shear rate conditions are retained. A key parameter is the time required for the high viscosity to recover after shearing. Long recovery times mean that after printing, a structure will continue to flow, losing resolution and integrity. As shown in FIG. 6, hydrogels doped with X0 and X20 glass particles behaved like undoped hydrogels. Their long recovery times (60 and 90 seconds, respectively) made it difficult to build 3D structures with the desired resolution. The viscosities of hydrogels doped with X40 and X60 glass particles, on the other hand, recovered almost immediately after shearing, and so could be used to print structures with better resolution. Recovery time is directly related to glass dissolution rate and likely Ca²⁺ion release, and it is also related to how a filament behaves after printing.

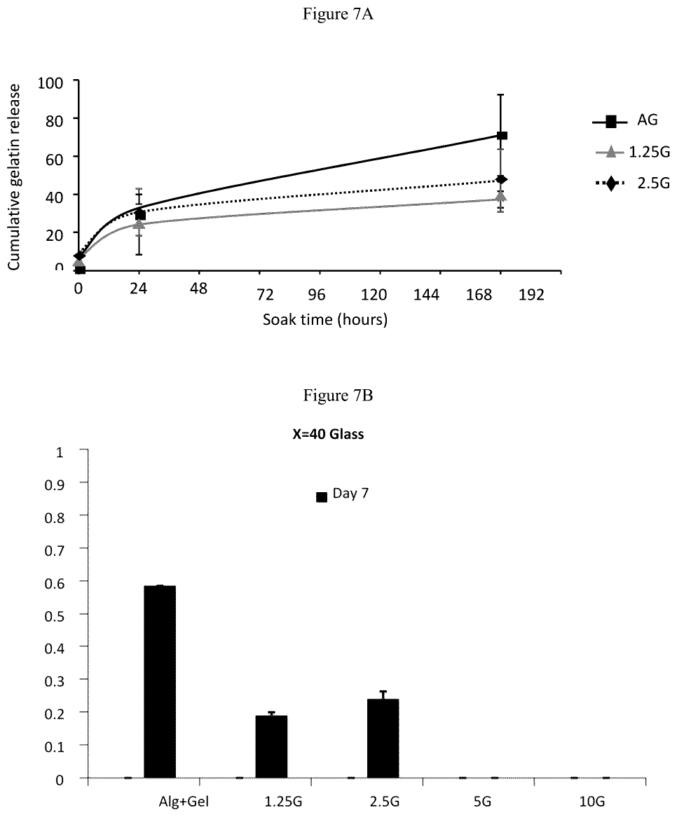

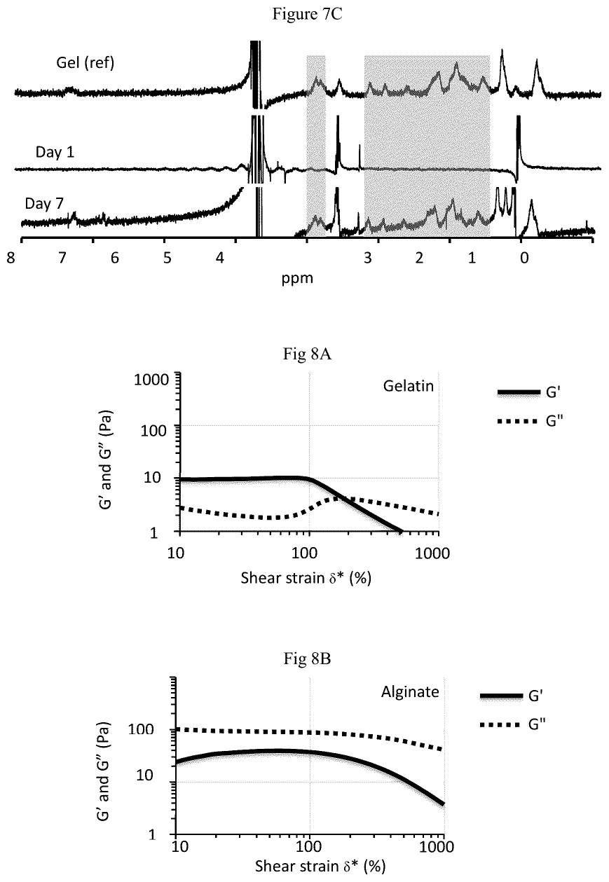

An additional benefit of alginate crosslinking by the controlled release of Ca-ions from the borophosphate glass particles was the reduced rate at which gelatin was released from the printed scaffolds. This is shown, for example, in FIGS. 7A-7C. The gelatin-fractions from undoped scaffolds dissolved in a matter of hours in water, whereas little gelatin was released from hydrogels doped with 5 and 10 wt. % X40 samples (FIGS. 7A-7C).

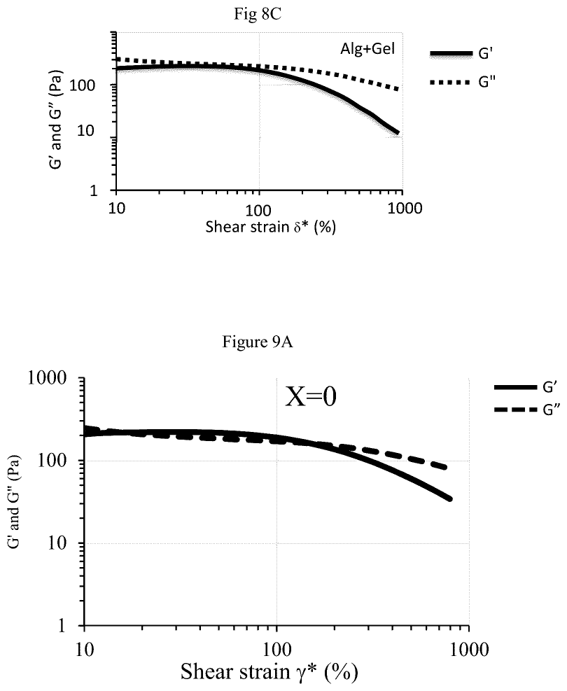

Other rheological methods that have bioprinting implications are simulated by oscillatory tests which characterize the viscoelastic features of hydrogel inks. In FIGS. 8A-8C, the viscous modulus (G″-grey) and the elastic modulus (G′-red) are reported. G″ is used to describe how the ink passes through a tip and G′ is linked to precision during printing and the shape fidelity post printing. The ratio of G′ (red) and G′′ (grey) represent the different behaviors, which are: elastic (G′>G′′), viscous (G′′>G′), and viscoelastic is when elastic behavior transitions to viscous. FIG. 8A depicts the results for gelatin hydrogels. FIG. 8B depicts the results for alginate hydrogels. FIG. 8C depicts the results for alginate-gelatin hydrogels.

FIGS. 8A-8C indicate that only gelatin shows viscoelastic behavior until about 200 shear strain, while alginate and alginate-gelatin samples only exhibit viscous behavior over the shear strain range. Even though gelatin is viscoelastic, the pressure needed is extremely low compared to the other two groups, this results in an inability to retain filament shape post print. Elastic properties are able to influence the extrusion pressure of the hydrogel ink and is co-determined by G′ and G″.

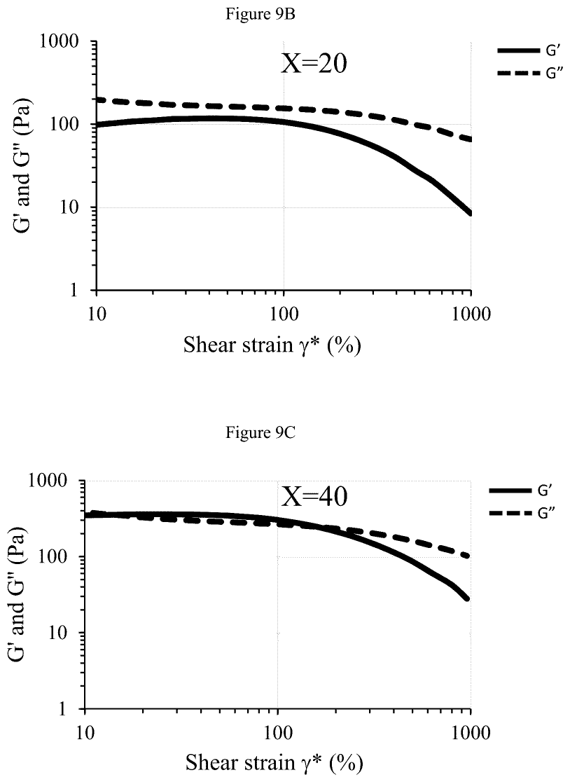

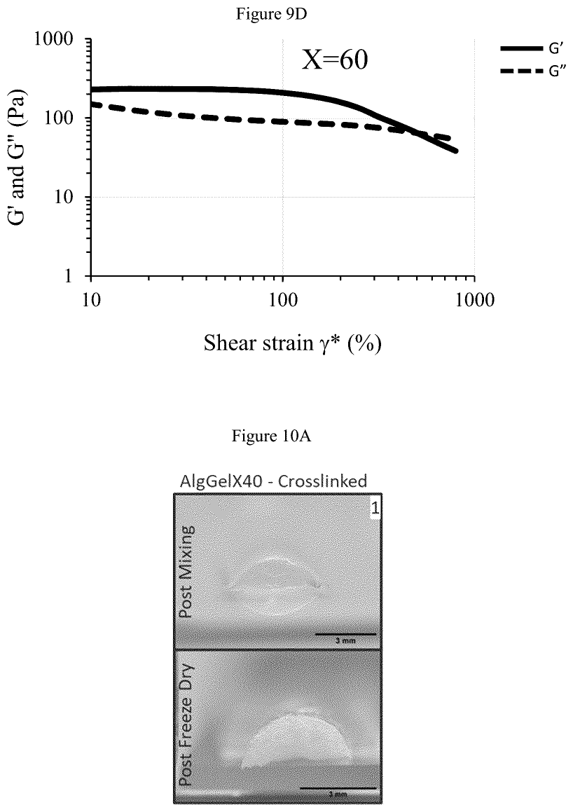

FIGS. 9A-9D show the elastic or viscous behavior of the hydrogel with the different borophosphate glass particles. FIG. 9A depicts the results for hydrogels with X0. FIG. 9B depicts the results for hydrogels with X20. FIG. 9C depicts the results for hydrogels with X40. FIG. 9D depicts the results for hydrogels with X60.

Only X40 and X60 displayed viscoelastic behavior. X40 had elastic characteristics until about 200% shear strain and X60 over 300% shear strain, while X20 and X0 displayed viscous behavior comparable to alginate and alginate-gelatin hydrogels, respectively. These results also correspond to the glass particle dissolution rate of the borophosphate glasses discussed above. Viscosity itself, which is usually considered the primary physicochemical parameter that affects print quality, does not reflect how complex the behavior of hydrogels is during the 3D printing process.

For each of the above rheological tests, five samples in each set were used. The results were reported as average±standard deviation. Minitab® software was used to analyze the difference in means of different groups using One-way ANOVA. The means were considered significantly different if the P-value was less than 0.05.

### Example 4: Comparison of X40 and Crosslinking on Hydrogel Microstructure

Further analysis of the microstructure of the hydrogels comprising borophosphate glass particles discussed above was conducted.

Alginate-gelatin hydrogels comprising borophosphate glass particles were extruded by 10 ml syringe on a microslip with a size around 2 mm diameter and 1 cm in length. To prepare the extruded samples for scanning electron microscopy (SEM), the samples were subjected to a number of steps. Samples were first put in a −20° C. freezer for 24 hrs after printing/crosslinking, then put in a FreeZone 2.5 Liter −50° C. Benchtop Freeze Dryer (commercially available from Labconco, Kansas City, MO) for 24 hrs. The free dried samples were then warmed to room temperature, taken to the Advanced Materials Characterization Laboratory (Missouri S&T, Rolla, MO), and broken in half (LN2 was used to help break the alginate-gelatin crosslinked sample). A PrismaE Scanning Electron Microscope (commercially available from Thermo Fisher Scientific Inc.) was used to analyze the interior cross section of each sample.

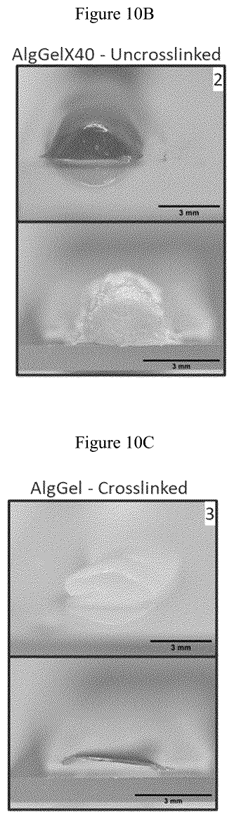

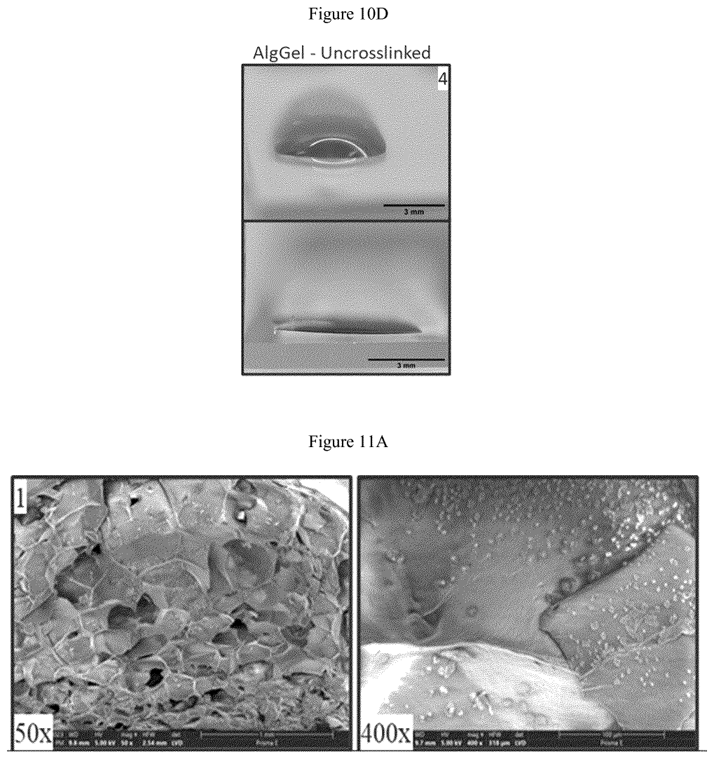

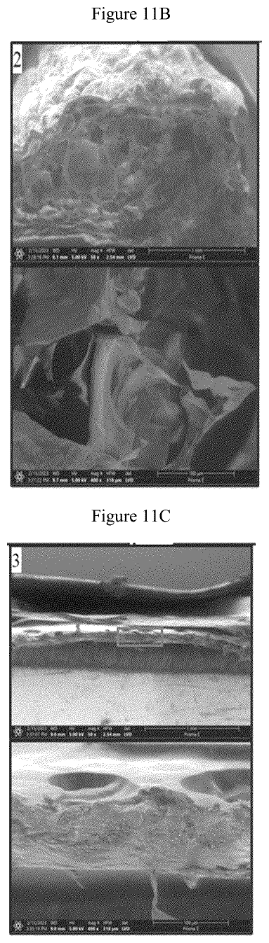

It was decided to examine how the different glass particles affected the hydrogel shape during the process. Samples were imaged by a digital single-lens reflex camera (DSLR) (Nikon D5500 with a AF-S DX Micro NIKKOR 40 mm f/2.8 G lens, Nikon Inc, USA) at various points in this process. Images were taken: after extrusion, after being extruded and subjected to −20° C. for 24 hours, after being extruded and freeze dried, and after observation using SEM. Dorsal and lateral images were taken of four samples per group. These images are reproduced in FIGS. 10A-10D, with a post mixing sample shown on top and a post freeze dry sample shown on bottom. FIG. 10A depicts a crosslinked (with 0.1 M CaCl₂) alginate-gelatin hydrogel with X40 glass particles. FIG. 10B depicts an uncrosslinked alginate-gelatin hydrogel with X40 glass particles. FIG. 10C depicts a crosslinked alginate-gelatin hydrogel without glass particles. FIG. 10D depicts an uncrosslinked alginate-gelatin hydrogel without glass particles.

Both samples with X40 were able to retain their shape under the pressure of the vacuum during freeze drying (FIGS. 10A-10B), while both groups without glass particles collapsed (FIGS. 10C-10D).

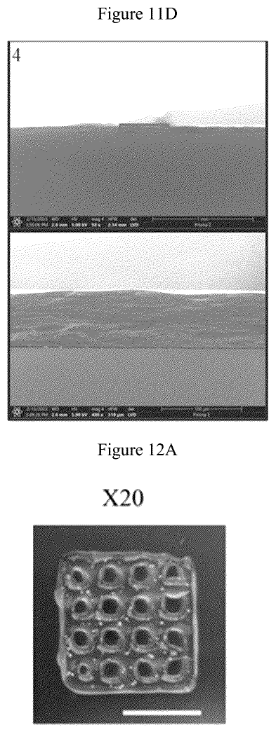

SEM images of the hydrogel with or without X40 and with or without crosslinking, shown in FIGS. 11A-11D, indicate that glass particles influence the microstructure of the hydrogel during freeze drying. The SEM images depict microstructure with and without crosslinker by Environmental SEM at 50× and 400× magnification. FIG. 11A depicts a crosslinked alginate-gelatin hydrogel with X40 glass particles. FIG. 11B depicts an uncrosslinked alginate-gelatin hydrogel with X40 glass particles. FIG. 11C depicts a crosslinked alginate-gelatin hydrogel without glass particles. FIG. 11D depicts an uncrosslinked alginate-gelatin hydrogel without glass particles. The rectangle indicates the sample in 50× magnification.

Only alginate-gelatin samples with X40 (FIGS. 11A-11B) still retained their shape after freeze drying and had a microstructure with cells, but only sample 1 (FIG. 11A) that was crosslinked had any visible pores. Meanwhile, samples 3 (FIG. 11C) and 4 (FIG. 11D), which did not contain glass particles, collapsed with no visible microstructure. This collapse and absence of visible microstructures was surprising for alginate-gelatin crosslinked by CaCl₂.

### Example 5: Cell Viability with Bio-Ink and Glass Particles

### Cell Culture

ASCs (adipose stem cells) were prepared by thawing frozen vials of approximately 1×10⁶ cells (commercially available from Obatala Sciences, LLC, New Orleans, LA) into 150 cm² culture plates (commercially available from Nunc, Rochester, NY) in 20 mL complete culture media (CCM) consisting of alpha minimum essential media (α-MEM; commercially available from Sigma-Aldrich, St. Louis, MO), 10% fetal bovine serum (FBS; commercially available from VWR, Dixon, CA), 1%100× L-glutamine (commercially available from Sigma-Aldrich), and 1%100× antibiotic/antimycotic (commercially available from Sigma-Aldrich). After 24 hours of incubation at 37° C. humidified and 5% CO₂, media was removed and the adherent, viable cells were washed twice with phosphate buffer solution (PBS; commercially available from Sigma-Aldrich) and harvested using 0.25% trypsin/1 mM Ethylenediaminetetraacetic acid (EDTA; commercially available from Sigma-Aldrich). ASCs then were plated at 100 cells/cm² in CCM. The media was changed every 3-4 days, and sub-confluent cells (≤70% confluent) from three separate donors between passages 2-6 were used for all experiments.

### Scaffold Fabrication

ASCs were added to the hydrogel ink after the alginate hydorogel had finished mixing. A 4×10⁶ ASCs pellet was re-suspended in 0.2 mL CCM, pipetted into a alginate-gelatin hydrogel, and magnetically stirred for no more than 3 min to obtain a uniform cell distribution and a final ASC concentration of 1.0×106 cells per 1 mL of bio-ink. The bio-ink was then transferred to the 3 mL syringes and made ready for bioprinting. A custom-modified tabletop cartesian 3D printer including syringes connected through digital syringe dispenser (Loctite®, commercially available from Rocky Hill, CT, USA) was used to fabricate scaffolds. Scaffold dimensions were set to 15 mm length, 15 mm width, and ˜1 mm thick (6 layers), and scaffolds were printed with 0-90° filament orientation in alternate layers. A customized software was written for g-code generation and syringe dispenser control. Sterile practices were followed for scaffold fabrication with ASCs, bio-ink syringes were maintained at room temperature, and the scaffolds were bioprinted in less than an hour inside the laminar flow hood.

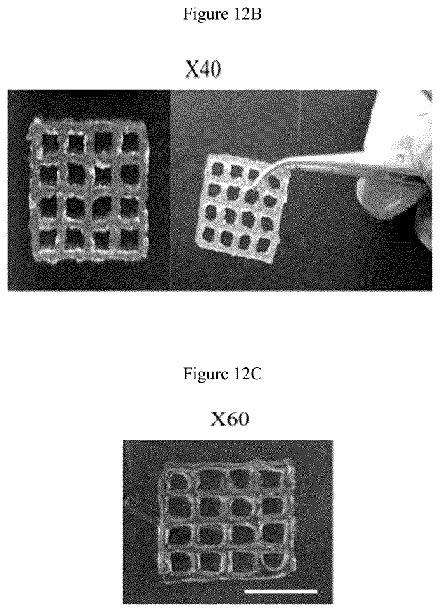

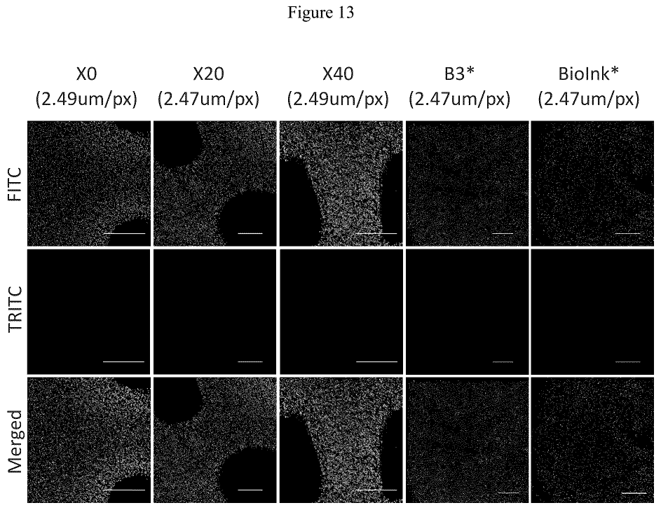

Scaffolds were fabricated with hydrogels containing borophosphate glass particles, to observe the printability of each glass type. Grid scaffolds were printed from hydrogels doped with X20 glass particles (FIG. 12A), X40 glass particles (FIG. 12B), and X60 glass particles (FIG. 12C).

X40 and X60 displayed good printability as seen by the square pores and generally constant filament size. Hydrogels with X0 and X20 glass particles behaved like a standard hydrogel. The printability of X20 was poor compared to X40 and X60. The ink did not retain its shape post print and caused the pores to become irregular circles, and there was no clear filament observed. This corresponds to the results from the rheology tests above, where X40 and X60 had instant hydrogel recovery and higher viscosity during shear stress. The dwell time for each scaffold was adjusted based on the hydrogel recovery.

### Cell Viability

Cell viability was evaluated using a Live/Dead kit (commercially available from ThermoFisher Scientific Inc., USA), according to the manufacturer's protocol. Scaffolds were prepared from hydrogels doped with 10 wt. % of borophosphate glass particles and infused with ASCs. Scaffolds were washed with PBS and soaked in 1 mL of prepared reagent (Calcein AM to stain live cells green and Ethidium homodimer-1 to stain dead cells red) for 30 min at room temperature. Micrographs were taken with 10× objective on a Nikon A1R-HD Eclipse Ti2 inverted confocal microscope (commercially available from Nikon Instruments Inc., USA). Three scaffolds were examined per experimental group, and images were taken covering an area of 6×6 mm² of the full scaffold thickness. A Z-step thickness of 40 μm was used during image acquisition. One maximum intensity projection image of multiple Z-step images was used for quantification. Cell viability was calculated as [live cells/(live cells+dead cells)]×100%. Live/Dead images were quantified using Fiji ImageJ software. Cell viability was determined immediately after printing the scaffold and then after one and seven days.

Three samples in each set were used for cell viability quantification. The results were reported as average±standard deviation. Minitab® software was used to analyze the difference in means of different groups using One-way ANOVA. The means were considered significantly different if the P-value is less than 0.05.

FIG. 13 shows the live and dead cells in representative hydrogel samples doped with 10wt. % X0, X20, X40, and X60 glass particles. A greater percentage of dead cells (bottom row) was observed in hydrogels doped with X20 and X60 glass particles, whereas few dead cells were found with the hydrogel samples prepared with X40 glass particles.

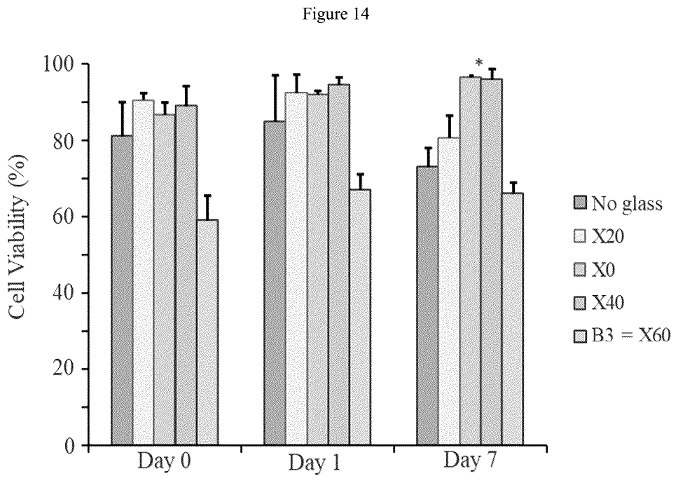

The quantitative cell viability analyses are summarized in FIG. 14. Over 80% of the ASCs in hydrogels doped with acidic (X0, X20) and PH neutral (X40) glass particles remained viable after printing, whereas only 60% of the ASCs remained viable in hydrogels doped with basic (X60) glass particles. After seven days, over 90% of the cells remained viable in hydrogels doped with X0 and X40. This compared favorably with the undoped hydrogels, where 75% of the cells were viable after seven days and the X60 glass particles, where only 65% of the cells remained viable. Cell viability with several of the 10 wt. % borophosphate glass particles after seven days was statistically greater than for the undoped alginate-gelatin system, indicating that the glass particles indeed promoted cell viability. The results for the basic X60 glass particles are consistent with those reported in the literature; as little as 1 wt. % loading of a basic bioactive silicate glass has been reported to be sufficient to reduce cell viability in an alginate-gelatin bio-ink, as did 2.5 wt. % of the basic bioactive borate glass 13-93B3. Thus, the fast release of boron ions proved toxic to cells, while pH neutral X40 glass particles provided an excellent environment for cells because of its controlled ion release. X0 also provided a good environment despite its low pH.

When introducing elements of the present disclosure or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the disclosure are achieved and other advantageous results attained.

As various changes could be made in the above products and methods without departing from the scope of the disclosure, it is intended that all matter contained in the above description and the associated drawings shall be interpreted as illustrative and not in a limiting sense.

What is claimed is:

1. A bio-ink comprising:
alginate,
gelatin, and
borophosphate glass particles, wherein the borophosphate glass particles have a nominal molar composition of 16Na₂O-24CaO-xB₂O₃-(60-x)P₂O₅ (mol %), wherein x is an integer from 0 to 60.

2. The bio-ink of claim 1, wherein the bio-ink further comprises mammalian cells.

3. The bio-ink of claim 1, wherein the bio-ink further comprises a crosslinker.

4. The bio-ink of claim 1, wherein x is from about 5 to about 60, from about 10 to about 60, from about 15 to about 60, from about 20 to about 60, from about 25 to about 60, from about 30 to about 60, from about 30 to about 55, from about 30 to about 50, from about 30 to about 45, or from about 30 to about 40.

5. The bio-ink of claim 1, wherein the borophosphate glass particles are from about 10 μm to about 500 μm, from about 30 μm to about 400 μm, from about 50 μm to about 300 μm, from about 50 μm to about 200 μm, from about 75 μm to about 150 μm, or from about 100 μm to about 125 μmin diameter.

6. The bio-ink of claim 1, wherein the pH is from 2 to about 6, from about 3 to about 6, from about 4 to about 6, from about 5 to about 6, or from about 5.5 to about 6.

7. The bio-ink of claim 1, wherein the gelatin is from about 0.1% to about 15 w/v %, from about 0.5% to about 12 w/v %, from about 1% to about 10 w/v %, from about 2 to about 7 w/v %, from about 2 to about 5 w/v %, or from about 3 to about 4 w/v % of the bio-ink, wherein w/v % is weight per volume of total bio-ink.

8. The bio-ink of claim 1, wherein the alginate is from about 0.1% to about 15 w/v %, from about 0.5% to about 12 w/v %, from about 1% to about 10 w/v %, from about 2 to about 7 w/v %, from about 2 to about 5 w/v %, or from about 3 to about 4 w/v % of the bio-ink, wherein w/v % is weight per volume of total bio-ink.

9. The bio-ink of claim 1, wherein borophosphate glass particles are from 0.1% to about 20 w/v %, from about 1% to about 20 w/v %, from about 2% to about 18 w/v %, from about 4% to about 16 w/v %, from about 6% to about 14 w/v %, from about 8 to about 12 w/v %, or from about 9% to about 11 w/v % of the bio-ink, wherein w/v % is weight per volume of total bio-ink.

10. The bio-ink of claim 1, wherein the bio-ink further comprises a solvent.

11. A method of preparing a bio-ink comprising:
a) dissolving gelatin in an aqueous solution;
b) adding borophosphate glass particles, wherein the borophosphate glass particles have a nominal molar composition of 16Na₂O-24CaO-xB₂O₃-(60-x)P₂O₅ (mol %), wherein x is an integer from 0 to 60 to the aqueous solution;
c) stirring the aqueous solution containing borophosphate glass particles until at least a portion of the borophosphate glass particles are suspended in the aqueous solution;
d) adding alginate to the aqueous solution having borophosphate glass particles suspended therein; and
e) mixing the aqueous solution until at least a portion of the alginate is suspended in the aqueous solution.

12. The method of claim 11, further comprising step f) wherein mammalian cells are added to the aqueous solution.

13. A bio-ink prepared according to the method of claim 11.

14. A method of printing a three-dimensional scaffold, the method comprising:
a) loading the bio-ink of claim 1 into a syringe barrel;
b) centrifuging the syringe until all air pockets are removed; and
c) extruding the bio-ink from the syringe into a three-dimensional scaffold with a desired shape.

15. The method of claim 14, wherein step c) is performed using a 3D printer.

16. The method of claim 14, wherein the three-dimensional scaffold comprises multiple layers, wherein adjacent layers are printed in orthogonal orientation to each other.

17. The method of claim 14, wherein the three-dimensional scaffold comprises from about one to about fifty layers, from about one to about twenty layers, from about five to about ten layers, or about six layers.

18. The method of claim 14, wherein the three-dimensional scaffold comprises one or more holes.

19. The method of claim 14, wherein the major axis of the desired shape is from about 0.1 mm to about 100 mm, from about 0.5 mm to about 50 mm, from about 1 mm to about 25 mm, or from about 5 mm to about 20 mm.

20. A three-dimensional scaffold for biomedical applications prepared according to the method of claim 14.
